(19) United States
(12) Patent Application Publication
(10) Pub. No.: US 2003/0083338 A1
(43) Pub. Date: May 1, 2003

(54) Compositions and methods for management of serotonin-mediated disorders

(76) Inventors: Mark G. Currie, Sterling, MA (US); Tom Jerussi, Framingham, MA (US); Paul Rubin, Sudbury, MA (US); Chris H. Senanayake, Shrewsbury, MA (US)

Correspondence Address: ROPES & GRAY, ONE INTERNATIONAL PLACE, BOSTON, MA, 02110-2624, US

(21) Appl. No.: 10/087,596

(22) Filed: Mar. 1, 2002

Related U.S. Application Data
(60) Provisional application No. 60/273,113, filed on Mar. 2, 2001.
(60) Provisional application No. 60/306,939, filed on Jul. 20, 2001.

(51) Int. Cl. A61K 31/496; A61K 31/165

(52) U.S. Cl. 514/254.05; 514/617

(57) ABSTRACT

The present invention provides methods and compositions for conjoint administration of a nefazodonoid and a fluoxetinoid for the treatment of depression and other neurological conditions.

### REFERENCE TO RELATED APPLICATIONS

This application claims priority to U.S. Provisional Patent Application No. 60/273,113 filed on Mar. 2, 2001 and to U.S. Provisional Patent Application No. 60/306,939 filed on Jul. 20, 2001, which are incorporated by reference herein.

### BACKGROUND OF THE INVENTION

“Depression” is a mood disorder that requires pharmacological treatment. The disorder is an expanding area of concern in the health care system. Studies on depression from Europe and the U.S. estimate a lifetime prevalence in females to be twice as high (9-26%) as in males (5-12%). There may be a genetic component to susceptibility; a first-degree degree relative of a depressed individual has an increased risk (1.5-3 times) of having depression.

Nefazodone (SERZONE®), 2-[3-[4-(3-chlorophenyl)-1-piperazinyl]-propyl-]5-ethyl-2,4-dihydro-4-(2-phenoxy-ethyl)-3H-1,2,4-triazol-3-one, is an antidepressant chemically unrelated to tricyclic or tetracyclic antidepressants and the selective serotonin uptake inhibitors in current use. Nefazodone has at least two activities in vivo. It blocks serotonin 5-HT₂ receptors at low doses and reversibly inhibits serotonin re-uptake at higher doses. It does not inhibit monoamine oxidase and exhibits decreased anticholinergic, antihistamine, alpha-adrenergic and sedative activity relative to tricyclic antidepressants. At low doses (e.g., 20-40 mg/day in adult humans), nefazodone selectively inhibits 5-HT_{2.} However, clinically useful effects typically require much higher doses (e.g., 300-600 mg/day) at which serotonin reuptake is also inhibited. Davis et al., Drugs 1997, 53, 608-636; Sanchez et al., Cell Mol. Neurobiol. 1999, 19, 467-489.

Nefazodone exhibits a significant first-pass metabolism, with the result that the immediate-release tablets show a bioavailability of approximately 20% and significant levels of three pharmacologically-active metabolites, a triazoledione, hydroxynefazodone and m-chlorophenylpiperazine (mCPP). It has been suggested that the metabolite mCPP may be responsible for some of the undesirable effects associated with nefazodone administration. The metabolite mCPP is a partial agonist at the 5-HT_{2B} and 5-HT_{2C} receptors and has some antagonist activity at 5-HT_{2A} receptors. In rodents it has anxiogenic-like properties, causes hypoactivity, hypophagia, oral dyskinesia, penile erection and hyperthermia. A dose-dependent hypoglycemic effect of mCPP mediated through 5-HT_{2C} or 5HT_{2B} receptors is seen in rats. It has been shown to increase anxiety in humans (and can cause panic attacks), may precipitate migraine attacks in those susceptible to such attacks, can disrupt sleep, be hypophagic in humans, and may have psychotogenic effects. As many of these effects are antagonistic to the beneficial effects of nefazodone, and because some of the described effects of mCPP are reminiscent of some of the adverse effects of nefazodone, a strategy for reducing the plasma levels of mCPP while retaining the effectiveness of nefazodone is desirable.

### SUMMARY OF THE INVENTION

The present invention relates, in part, to a method for providing a low dose nefazonoid treatment that may reduce certain of the side effects occurring at traditional higher doses.

In certain embodiments, the present invention makes available pharmaceutical preparations comprising a nefazodonoid, a serotonin reuptake inhibitor (SRI), and a pharmaceutically acceptable excipient. In certain embodiments, the SRI is substantially commingled with the nefazodonoid and the excipient, while in other embodiments, the SRI and nefazodonoid are present in distinct areas of the preparation, e.g., different layers of a pill, different halves of a capsule, etc. In certain embodiments, the nefazodonoid is nefazodone, hydroxynefazodone, or a mixture of the two. In certain embodiments, the SRI is selected from fluoxetine, norfluoxetine, and a combination thereof, any of which may be present in a racemic form, while in other embodiments, the SRI is enriched for one isomer, e.g., the S-isomer or, preferably, the R-isomer. In certain embodiments, the pharmaceutical preparation of the present invention is suitable for oral administration, e.g., in the form of a pill, tablet, capsule, or other solid formulation. In certain embodiments, the SRI and the nefazodonoid are chemically linked, e.g., through a linkage capable of being cleaved under biological conditions.

In one aspect, the present invention provides a pharmaceutical preparation comprising a nefazodonoid and an SRI, and more preferably a selective serotonin reuptake inhibitor (SRI), in a pharmaceutically acceptable excipient. In certain embodiments, the nefazodonoid is selected from nefazodone, hydroxynefazodone, oxonefazodone, a mixture thereof, and a pharmaceutically acceptable salt thereof, e.g., R-hydroxynefazodone. In certain embodiments, the SRI is an SSRI, such as a fluoxetinoid, e.g., fluoxetine and norfluoxetine, including R-fluoxetine.

In certain embodiments, the pharmaceutical preparation is formulated for oral administration. In certain embodiments, the nefazodonoid and SRI are commingled in single dosage form. In certain embodiments, the nefazodonoid and SRI are provided in separate dosage form. In certain embodiments, the nefazonoid is provided in an amount, for single dosage, sufficient to provide the ED₅₀ for 5-HT receptor inhibition, but less than half the ED₅₀ for inhibition of serotonin reuptake. In certain embodiments, the SRI is provided in an amount, for single dosage, sufficient to provide the ED₅₀ for inhibition of serotonin reuptake, but less than half the ED₅₀ for 5-HT receptor inhibition.

In another aspect, the invention provides a pharmaceutical preparation comprising, in a single dosage form, a mixture of a nefazodonoid and an SRI. In certain embodiments, the nefazodonoid is selected from nefazodone, hydroxynefazodone, and oxonefazodone. In certain embodiments, the single dosage form contains from 10-100 mg nefazodone, oxonefazodone, or hydroxynefazodone. Exemplary embodiments include single dosage forms of equal to or less than 50, 40, 25, 20 or even 10 mg. In certain embodiments, the SRI is selected from fluoxetine and norfluoxetine. In certain embodiments, the single dosage form contains from 5-40 mg fluoxetine or norfluoxetine equal to or less than, e.g., 20, 15, 10 or even 5 mg.

In yet another aspect, the invention provides a kit comprising, in single dosage form, a nefazodonoid and a selective serotonin reuptake inhibitor, each in a pharmaceutically acceptable excipient; and instructions for co-administering the nefazodonoid and a selective serotonin reuptake inhibitor in a treatment of a serotonin-mediated disorder.

In still another aspect, the invention relates to a method for treating a 5-HT receptor-mediated disorder in an animal, comprising co-administering (e.g., simultaneously or at different times) to the animal an amount of a nefazodonoid sufficient to inhibit a 5-HT₂ receptor activity to a therapeutically effective extent, and an amount of an SRI sufficient to inhibit serotonin reuptake to a therapeutically effective extent, such that the nefazodonoid is administered at a dosage below the necessary dosage to inhibit serotonin reuptake to a therapeutically effective extent in the absence of the SSRI. In certain embodiments, the nefazodonoid and the SSRI are administered simultaneously. In certain embodiments, the nefazodonoid and the SSRI are administered as part of a single composition. In certain embodiments, the single composition is for oral administration. In certain embodiments, the nefazodonoid is selected from nefazodone, hydroxynefazodone, oxonefazodone, a mixture thereof, and pharmaceutically acceptable salts thereof. such as R-hydroxynefazodone. In certain embodiments, the SSRI is a SRI or a pharmaceutically acceptable salt thereof, e.g., is selected from fluoxetine and norfluoxetine, such as R-fluoxetine, or a mixture thereof.

In still another aspect, the invention provides a method for treating depression in a human patient, comprising administering to the patient (a) a nefazodonoid selected from nefazodone, hydroxynefazodone, or oxonefazodone in an amount of 100 mg or less per day, and (b) a SRI, such as selected from fluoxetine or norfluoxetine in an amount sufficient to inhibit serotonin reuptake to a therapeutically effective extent. In certain embodiments, the nefazodonoid and the SRI are administered to the patient simultaneously. In certain embodiments, the SRI is administered at a rate of 5-40 mg per day.

In yet another aspect, the invention relates to a method for preparing a pharmaceutical preparation, comprising combining a nefazodonoid, a SRI, and a pharmaceutically acceptable excipient in a composition for simultaneous administration of the nefazodonoid and the SRI.

In yet a further aspect, the invention provides a pharmaceutical preparation of a nefazodonoid and a fluoxetinoid for use in the treatment of a 5-HT receptor mediated disorder.

In still another aspect, the invention relates to a method for conducting a pharmaceutical business, by manufacturing a preparation of any of claims 1-9 or a kit of claim 17, and marketing to healthcare providers the benefits of using the preparation or kit in the treatment of 5-HT receptor-mediated disorders.

In yet another aspect, the invention provides a method for conducting a pharmaceutical business, by providing a distribution network for selling the preparation of any of claims 1-9, and providing instruction material to patients or physicians for using the preparation to treat 5-HT receptor-mediated disorders.

In still a further aspect, the invention relates to a method for conducting a pharmaceutical business, by determining an appropriate formulation and dosage of a nefazodonoid and a selective serotonin reuptake inhibitor to be co-administered in the treatment of a 5-HT receptor mediated disorder, conducting therapeutic profiling of identified formulations for efficacy and toxicity in animals, and providing a distribution network for selling a preparation as having an acceptable therapeutic profile. In certain embodiments, the method further includes an additional step of providing a sales group for marketing the preparation to healthcare providers.

In yet another aspect, the invention provides a method for conducting a pharmaceutical business by determining an appropriate formulation and dosage of a nefazodonoid and a selective serotonin reuptake inhibitor to be co-administered in the treatment of a 5-HT receptor mediated disorder, and licensing, to a third party, the rights for further development and sale of the formulation.

Still another aspect provides a low-dose formulation of a fluoxetinoid, such as such as selected from fluoxetine or norfluoxetine. Such formulations in include single dose formulations, such as for oral administration, of less than 5-40 mg, and more preferably 5-25, or even 5-10 mg. In certain preferred embodiments, the subject invention provides fluoxetinoid tablets, capsules or the like having 5-40mg of a fluoxetinoid.

### DETAILED DESCRIPTION OF THE INVENTION

### I. Overview

The present invention provides compositions and methods for treatment of serotonin-mediated disorders, particularly 5-HT₂ receptor-mediated disorders. In particular, the subject invention provides for combinatorial therapies in which nefazodone or an analog thereof (collectively herein a “nefazodonoid”) is co-administered with a selective serotonin reuptake inhibitor (SSRI), such as fluoxetine.

The ED₅₀ of nefazodone for serotonin re-uptake is significantly higher than that for 5-HT₂ receptor inhibition. In general, the art reports an optimum therapeutic dosage of nefazodone to be between 300 and 600 mg/day. See, for example, Davis et al. (1997) Drugs 53:608 “Nefazodone: a review of its pharmacology and clinical efficacy in the management of major depression”. Dosing patients with an amount of nefazodone sufficient to therapeutically inhibit both receptor activation and serotonin reuptake substantially increases the risk of unwanted side effects encountered during high dose administration of nefazodone. By combining nefazodone treatment with the use of more potent SSRIs, nefazodone can be administered at doses substantially lower than 300-600 mg/day, thereby avoiding side-effects of high dose nefazodone treatment, yet still achieving the same desired therapeutic effect resulting from 5-HT receptor antagonism and serotonin reuptake inhibition. For example, the subject method can be practiced by administering nefazodone at a dosage rate high enough to therapeutically inhibit 5-HT₂ receptor activity, e.g., to reach the ED₅₀ for receptor inhibition, yet less than the ED₅₀ for serotonin reuptake. In certain embodiments, the nefazodone dosage is an amount which produces less than one half the ED₅₀ for serotonin reuptake, and more preferably less than one tenth the ED₅₀ for serotonin reuptake.

Likewise, it is specifically contemplated that the dosage of the co-administered SSRI can be reduced relative to treatment protocols in which SSRIs are used alone. The potency of fluoxetine, to illustrate, is complementary to that of nefazodone in that it inhibits 5-HT receptor activity with an ED50 five to ten times greater than its ED50 for inhibiting serotonin reuptake. Combined with a nefazonoid, the dosage of the SSRI can be adjusted to reach the ED50 for inhibiting serotonin reuptake, yet remain below the ED50 for 5-HT receptor antagonism.

In the present method, by employing doses of each compound that are lower than would be required to achieve full therapeutic benefits by either used alone, the present invention provides for the treatment of neurological conditions, such as depression, while minimizing unwanted side effects normally associated with high doses of nefazodoids or SSRIs. For example, nefazodone has been shown to antagonize alphal-adrenergic receptors. Blockade of alphal-adrenergic receptors produces sedation, muscle relaxation, and cardiovascular effects such as hypotension, reflex tachycardia, and minor changes in ECG patterns. These and other unwanted side effects such as headaches, nervousness, anxiety, insomnia, inner restlessness (akathisia), suicidal thoughts, self mutilation, manic behavior, nausea, diarrhea, drowsiness, decreased libido, and/or sexual dysfunction should be significantly lessened by employing the methods and compositions described herein in place of therapeutic dosages of either nefazodone or fluoxetine alone.

Moreover, while the present application may provide examples based on nefazodone and fluoxetine, it will be readily understood that the subject method and compositions are intended to be useful with other combinations of nefazonoids and SSRIs.

Accordingly, the present invention provides methods for treating neurological conditions such as depression by co-administering a nefazodonoid with an SSRI, pharmaceutical preparations including both the nefazodonoid and the SSRI, kits for coadministration including formulations of each of the nefazodonoid and the SSRI, and methods of preparing such pharmaceutical preparations.

### II. Definitions

For convenience, certain terms employed in the specification, examples, and appended claims are collected here.

The term “ED₅₀” means the dose of a drug which produces 50% of its maximum response or effect. Alternatively, the dose which produces a predetermined response in 50% of test subjects or preparations.

The term “LD₅₀” means the dose of a drug which is lethal in 50% of test subjects.

The term “therapeutic index” refers to the therapeutic index of a drug defined as LD₅₀/ED₅₀.

The term “structure-activity relationship (SAR)” refers to the way in which altering the molecular structure of drugs alters their interaction with a receptor, enzyme, etc.

The term “agonist” refers to a compound that mimics the action of natural transmitter or, when the natural transmitter is not known, causes changes at the receptor complex in the absence of other receptor ligands.

The term “antagonist” refers to a compound that binds to a receptor site, but does not cause any physiological changes unless another receptor ligand is present.

The term “ligand” refers to a compound that binds at the receptor site.

The term “psychotic condition” as used herein means pathologic psychological conditions which are psychoses or may be associated with psychotic features. Such conditions include, but are not limited to the psychotic disorders which have been characterized in the DSM-IV-R, Diagnostic and Statistical Manual of Mental Disorders, Revised, 4th Ed. (1994), including schizophrenia and acute mania. The DSM-IV-R was prepared by the Task Force on Nomenclature and Statistics of the American Association, and provides clear descriptions of diagnostic categories. The skilled artisan will recognize that there are alternative nomenclatures, nosologies, and classification systems for pathologic psychological conditions and that these systems evolve with medical scientific progress.

The term “bipolar disorder” as used herein refers to a condition characterized as a Bipolar disorder, in the DSM-IV-R as category 296.xx, including both Bipolar Disorder I and Bipolar Disorder II.

The term “autistic disorder” as used herein means a condition characterized as an Autistic Disorder in the DSM-IV-R as category 299.xx, including 299.00, 299.80, and 299.10, preferably 299.00.

The term “anxiety disorder”0 includes, but is not limited to obsessive-compulsive disorder, psychoactive substance anxiety disorder, post-traumatic stress disorder, generalized anxiety disorder, anxiety disorder NOS, and organic anxiety disorder.

The term “excessive aggression” as used herein refers to a condition characterized by aggression that is so excessive that it interferes with the individual's daily functions, relationships, and may threaten the safety of the individual, for example in a situation in which violent suicide is contemplated. The excessive aggression which may be treated using the method claimed herein is independent of a psychotic condition and not directly related to the consumption of a drug or other substance.

The term “treating” as used herein includes prophylaxis of the named condition or amelioration or elimination of the condition once it has been established.

The term “heteroatom” as used herein means an atom of any element other than carbon or hydrogen. Preferred heteroatoms are boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

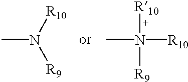

wherein R₉, R₁₀ and R′₁₀ each independently represent a hydrogen, an alkyl, an alkenyl, —(CH₂)ₘ-R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ-R_{8.} Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

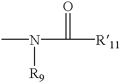

wherein R₉ is as defined above, and R′₁₁ represents a hydrogen, an alkyl, an alkenyl or —(CH₂)ₘ-R₈, where m and R₈ are as defined above.

The term “amido” is art recognized as an amino-substituted carbonyl and includes a moiety that can be represented by the general formula:

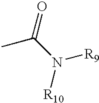

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ-R₈, wherein m and R₈ are defined above. Representative alkylthio groups include methylthio, ethyl thio, and the like.

The term “carbonyl” is art recognized and includes such moieties as can be represented by the general formula:

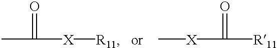

wherein X is a bond or represents an oxygen or a sulfur, and R₁₁ represents a hydrogen, an alkyl, an alkenyl, —(CH₂)ₘ-R₈ or a pharmaceutically acceptable salt, R′₁₁ represents a hydrogen, an alkyl, an alkenyl or —(CH₂)ₘ-R₈, where m and R₈ are as defined above. Where X is an oxygen and R₁₁ or R′₁₁ is not hydrogen, the formula represents an “ester”. Where X is an oxygen, and R₁₁₁ is as defined above, the moiety is referred to herein as a carboxyl group, and particularly when R₁₁₁ is a hydrogen, the formula represents a “carboxylic acid”. Where X is an oxygen, and R′¹¹ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —-O-alkyl, —O-alkenyl, —O-alkynyl, —O—(CH₂)ₘ-R₈, where m and R₈ are described above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as analgesics), wherein one or more simple variations of substituents are made which do not adversely affect the efficacy of the compound in binding to opioid receptors. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

### III. Exemplary Compositions

Nefazodonoids useful in the present methods and compositions include compounds that inhibit 5-HT₂ receptor activity and have a structure of the formula (I):

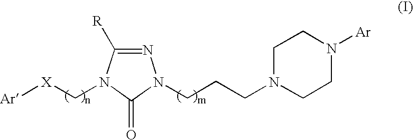

wherein, as valence and stability permit,

Ar and Ar′ represent, independently, substituted or unsubstituted aryl groups;

X represents O or S, preferably O;

R represents a hydroxyl or a substituted or unsubstituted lower alkyl group, lower alkoxy, lower acyloxy, aralkoxy, or aracyloxy group;

n represents an integer from 2-4, preferably 3; and

m represents an integer from 0-2, preferably 1.

In certain embodiments, Ar is phenyl group, and is unsubstituted or, preferably, is substituted with 1-5 substituents selected from halogen and CF₃ groups.

In certain embodiments, Ar′ is phenyl group, and is unsubstituted or, preferably, is substituted with 1-5 substituents selected from halogen and CF₃ groups.

In certain embodiments, R represents an ethyl group optionally substituted with a hydroxyl group, oxo group, or a lower acyloxy group. In embodiments wherein R represents hydroxyl, the formula is considered to include the triazoledione tautomer.

Examples of compounds which fall within, or can be modified with a hydroxyl, oxo, or other substituent to R in order to fall within, the above formula can be found in U.S. Pat. Nos. 4,338,317, 4,386,091, 4,613,600, 5,116,852, 4,575,555, and 4,487,773, and PCT publication WO 00/661128.

In certain embodiments, a nefazodonoid has a structure of the formula (II):

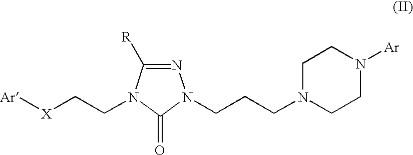

wherein, as valence and stability permit,

Ar and Ar′ represent, independently, phenyl rings, either unsubstituted or substituted with from 1-3 groups selected from halogen and CF₃ groups;

X represents O or S, preferably O; and

R represents a hydroxyl or a C₁-C₃ alkyl group, either unsubstituted or substituted with a hydroxyl, oxo, or lower acyloxy group.

In certain embodiments, Ar is unsubstituted or, preferably, is substituted with a halogen or CF₃ group.

In certain embodiments, Ar′ is unsubstituted or substituted with a halogen or CF₃, group.

In certain embodiments, R represents an ethyl group optionally substituted with a hydroxyl group. In embodiments wherein R represents hydroxyl, the formula is considered to include the triazoledione tautomer.

Nefazodone undergoes fairly rapid metabolism in the body, resulting in the formation of several metabolic derivatives. Of these, hydroxynefazodone and its oxonefazodone and triazoledione metabolic derivatives retain some or all of nefazodone's activity against 5-HT₂ receptors. Accordingly, any of these compounds as well as any other metabolic derivatives of nefazodone that retain some or all of nefazodone's 5-HT₂ inhibitory activity, and pharmaceutically acceptable salts of any of these, may be employed in the compositions and methods of the invention, and are considered to be nefazodonoids as the term is used herein.

The nefazodonoid is administered with a serotonin reuptake inhibitor (SRI). To illustrate, the SRI can be venlafaxine or a derivative thereof. For instance, the SRI can be a compound represented in Formula (IX), or a pharmaceutically acceptable salts thereof:

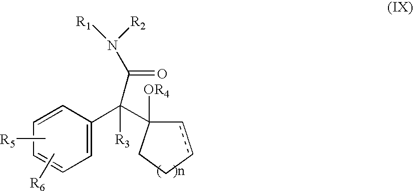

wherein

R₁ is hydrogen or alkyl of 1 to 6 carbon atoms;

R₂ is alkyl of 1 to 6 carbon atoms;

R₃ is hydrogen or alkyl of 1 to 6 carbon atoms;

R₄ is hydrogen, alkyl of 1 to 6 carbon atoms, formyl, or alkanoyl of 2 to 7 carbon atoms;

R₅ and R₆ are independently hydrogen, hydroxyl, alkyl of 1 to 6 carbon atoms, alkoxy of 1 to 6 carbon atoms, alkanoyloxy of 2 to 7 carbon atoms, cyano, nitro, alkylmercapto of 1 to 6 carbon atoms, amino, alkylamino of 1 to 6 carbon atoms, dialkylamino in which each alkyl group is of 1 to 6 carbon atoms, alkanamido of 2 to 7 carbon atoms, halo, trifluoromethyl, or, when taken together, methylene dioxy; and

n is one of the integers 0, 1, 2, 3 or 4.

The nontricyclic compound venlafaxine, chemically named (±)-1-[2-(dimethylamino)-1-(4-methoxyphenyl)ethyl]-cyclohexanol, is an antidepressant which has been studied extensively and which is described in, for example, U.S. Pat. No. 4,761,501 and Pento, J. T. Drugs of the Future 13(9):839-840 (1988).

Venlafaxine includes active derivatives of venlafaxine. The term “derivative” includes metabolites. Venlafaxine derivatives include: O-desmethylvenlafaxine and the single enantiomers of the two compounds.

In certain preferred embodiments, the venlafaxine compound is provided in optically pure form, such as optically pure (−)—N-desmethylvenlafaxine, chemically named (−)-1-[2-(methylamino)-1-(4-methoxyphenyl)ethyl]cyclohexanol; optically pure (−)—N,N -didesmethylvenlafaxine, chemically named (−)-1-[2-(amino)-1-(4-methoxyphenyl)ethyl]cyclohexanol; optically pure (−)—O-desmethylvenlafaxine, chemically named (−)-1-[2-(dimethylamino)-1-(4-phenol)ethyl]cyclohexanol; optically pure (−)—N,O-didesmethylvenlafaxine, chemically named (−)-1-[2-(methylamino)-1-(4phenol)ethyl]cyclohexanol; and optically pure (−)—O-desmethyl—N,N-didesmethylvenlafaxine, chemically named chemically named (-)-1-[2-(amino)-1-(4-phenol)ethyl]cyclohexanol.

In other embodiments, the SRI compound is optically pure a derivative of (+)-venlafaxine, such as (+)—O-desmethylvenlafaxine. U.S. Pat. No. 6197828 provides additional examples of derivatives of (+)-venlafaxine.

In preferred embodiments, the SRI is a selective serotonin reuptake inhibitor (SSRI). SSRIs include fluoxetinoids, sertraline (ZOLOFT), citalopram (CELEXA), paroxetine (PAXIL), and fluvoxamine (LUVOX), cericlamine, femoxetine, ifoxetine, cyanodothiepin, and litoxetine. The terms such as “sertraline,” “citalopram,” “paroxetine,” and “fluvoxamine” include active derivatives and metabolites, such as the demethyl metabolites norfluoxetine, demethylsertraline, and demethylcitalopram.

Preferred SSRIs are fluoxetinoids and citalopram (and its derivatives). More preferred SSRIs are fluoxetinoids.

Fluoxetinoids useful in the present methods and compositions include compounds that inhibit serotonin reuptake and have structures of the formula (III):

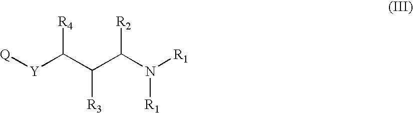

wherein, as valence and stability permit,

R₁, independently for each occurrence, represents H or lower alkyl, preferably H or Me;

R₂, R₃, and R₄ each independently represent H, methyl, substituted or unsubstituted phenyl, or substituted or unsubstituted phenylmethyl, such that exactly one of R₂, R₃, and R₄ is a substituted or unsubstituted phenyl, or substituted or unsubstituted phenylmethyl;

Y represents O, S, or —S(O)₂—, preferably O;

Q represents a substituted or unsubstituted aryl or heteroaryl ring, including polycyclic ring systems.

In certain embodiments, at least one occurrence of R₁ represents hydrogen.

In certain embodiments, R₂ and R₃ are selected from H and Me, preferably H, and R₄ represents a substituted or unsubstituted phenyl ring.

In certain embodiments, Q is a substituted or unsubstituted phenyl ring.

Examples of compounds which fall within the above formula can be found in U.S. Pat. Nos. 4,902,710, 4,824,868, 4,692,469, 4,626,549, 4,584,404 and 4,314,081.

In certain embodiments, a fluoxetinoid has a structure of the formula (IV):

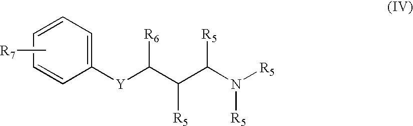

wherein, as valence and stability permit,

R₅, independently for each occurrence, represent H or Me;

R₆ represents a substituted or unsubstituted phenyl ring, preferably unsubstituted;

Y represents O, S, or —S(O)₂—, preferably O; and

R₇ represents from 1-5 substituents selected from halogen, lower alkyl, lower alkenyl, lower alkoxy, substituted or unsubstituted phenyl, and CF₃.

In certain embodiments, at least one occurrence of R₅ bound to N is a hydrogen.

In certain embodiments, R₆ represents an unsubstituted phenyl group.

In certain embodiments, R₇ represents from 1-2 substituents selected from halogen and CF₃.

Fluoxetine is metabolized far more slowly, with the primary metabolic derivative being norfluoxetine, which is similar to fluoxetine in selectivity and potency. Any combination of these compounds, racemic or enriched for either enantiomer, and pharmaceutically acceptable salts thereof may be employed in the methods and compositions described herein, and any one of these compounds is included in the term ‘fluoxetinoids’ as the term is used herein.

In certain embodiments, the SSRI is sertraline or a derivative thereof. For instance, the SSRI can be a compound represented in Formula (V), or a pharmaceutically acceptable salts thereof:

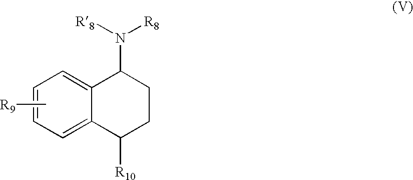

wherein

R₈ is selected from the group consisting of hydrogen and normal alkyl of from 1 to 3 carbon atoms;

R′₈ is normal alkyl of from 1 to 3 carbon atoms;

R₉ is selected from the group consisting of hydrogen, fluoro, chloro, bromo, trifluoromethyl and alkoxy of from 1 to 3 carbon atoms;

R₁₀ is

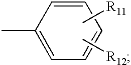

R₁₁ and R₁₂ are each independently selected from the group consisting of hydrogen, fluoro, chloro, bromo, trifluoromethyl, alkoxy of from 1 to 3 carbon atoms and cyano, with at least one of R₁₁ and R₁₂ being other than hydrogen; and

U.S. Pat. Nos. 4,536,518, 4,940,731, 4,962,128, and 5,130,338 describe sertraline and various derivatives and formulations thereof which can be used in the subject formulation and methods. Sertraline derivatives include N-desmethylsertraline.

In certain preferred embodiments, the compound is, as appropriate, the cis-isomeric base of formula (V). The term “cis-isomeric” refers to the relative orientation of the N(R′₈)R₈ and R₁₀ moieties on the cyclohexene ring (i.e. they are both oriented on the same side of the ring). Because both the 1 - and 4- carbons of the formula are asymmetrically substituted, each cis- compound has two optically active enantiomeric forms denoted (with reference to the 1-carbon) as the cis-(1R) and cis-(1S) enantiomers. The preferred embodiment is the (1S) enantiomer, e.g., cis-(1S)-N-methyl-4-(3,4-dichlorophenyl)-1,2,3,4-tetrahydro-1-naphthalenamine and its pharmaceutically acceptable acid addition salts.

In certain embodiments, the SSRI is paroxetine or a derivative thereof. For instance, the SSRI can be a compound represented in Formula (VI), or a pharmaceutically acceptable salts thereof:

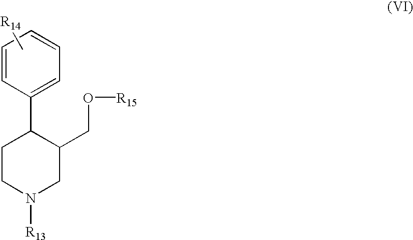

wherein

R₁₃ represents hydrogen or an alkyl group of 1-4 carbon atoms, and

R₁₄ represents hydrogen, alkyl having 1-4 carbon atoms, C1-6 alkoxy, C1-6 trifluoroalkyl (preferably, trifluoromethyl), hydroxy, halogen, methylthio, or C1-6 aryl(C1-6) alkyloxy (e.g., phenyl(C1-6)alkyloxy and benzyl(C1-6)alkyloxy), and

R₁₅ represents an alkyl or alkynyl group having 1-4 carbon atoms, or a phenyl group optionally substituted by C1-4 alkyl, C1-6 alkylthio, C1-6 alkoxy, halogen, nitro, acylamino, methylsulfonyl or methylenedioxy, or represents tetrahydronaphthyl.

In certain preferred embodiments, the SSRI is a compound represented in Formula (VI-A), or a pharmaceutically acceptable salts thereof:

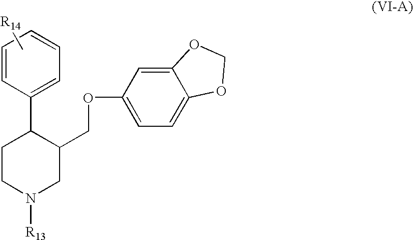

wherein R₁₃ represents hydrogen or an alkyl group of 1-4 carbon atoms, and R₁₄ is a halogen. In certain preferred embodiments, R₁₃ is a fluorine. Of particularly therapeutical effect is the (−) form of a compound of formula I, wherein R¹ is hydrogen and the fluorine is in para position.

The synthesis of paroxetine and of the acid addition salts thereof is described, inter alia, in U.S. Pat. No. 4,007,196 to Christensen et al. and U.S. Pat. No. 4,721,723 to Barnes et al. Derivative of paroxetine are also described in PCT publication WO035910.

In still other embodiments, the SSRI is citalopram or a derivative thereof. For instance, the SSRI can be a compound represented in Formula (VII), or a pharmaceutically acceptable salts thereof:

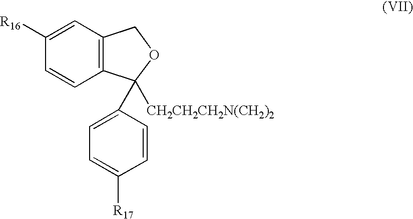

wherein R₁₆ and R₁₇ are each independently represent a halogen, a trifluoromethyl group, a cyano group or —C(═O)—R,₈, wherein R₁₈ is an alkyl radical with from 1-4 C-atoms inclusive.

Citalopram was first disclosed in DE 2,657,271 corresponding to U.S. Pat. No. 4,136,193. This patent publication describes the preparation of citalopram by one method and outlines a further method which may be used for preparing citalopra Methods of preparing the individual enantiomers of citalopram are disclosed in U.S. Pat. No 4,943,590, such as (+)-1-(3-Dimethylaminopropyl)-1-(4′-fluorophenyl)-1,3-dihydroisobenzofuran-5-carbonitrile. Citalopram derivatives include desmethylcitalopram and didesmethylcitalopram, and the single enantiomers of all three compounds.

In yet another embodiment, the SSRI is fluvoxamine or a derivative thereof. For instance, the SSRI can be a compound represented in Formula (VIII), or a pharmaceutically acceptable salts thereof:

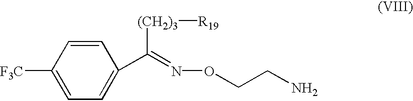

wherein R₁₉ represents a cyano group, a cyanomethyl group, a methoxymethyl group or an ethoxymethyl group. Fluvoxamine and other oxime ethers are disclosed in U.S. Pat. No. 4,085,225.

The magnitude of prophylactic or therapeutic doses of an SRI and a nefazodonoid will, of course, vary with the nature and the severity of the condition to be treated and the route of administration, as well as the age, weight and response of the individual patient. In general, the daily dose range of fluoxetine or norfluoxetine administered as part of the conjoint therapy contemplated herein lies within the range of from about 1 mg to about 100 mg per day, preferably about 5 mg to about 60 mg per day, and most preferably from about 10 mg to about 40 mg per day, in single or divided doses. In general, the daily dose range of nefazodone or hydroxynefazodone administered in conjoint therapy as contemplated herein lies within the range of from about 1 mg to about 100 mg per day, preferably about 5 mg to about 60 mg per day, and most preferably from about 10 mg to about 40 mg per day, in single or divided doses. On the other hand, it may be necessary to use dosages outside these limits in some cases.

Any suitable route of administration may be employed for providing the patient with effective dosages of an SRI and a nefazodonoid. For example, oral, rectal, parenteral, transdermal, subcutaneous, intramuscular, inhalation and the like may be employed. Dosage forms include tablets, troches, dispersions, suspensions, solutions, capsules, patches and the like.

The pharmaceutical compositions of the present invention comprise an SRI and a nefazodonoid as an active ingredient or a pharmaceutically acceptable salt thereof, and may also contain a pharmaceutically acceptable carrier and optionally other therapeutic ingredients. The term “pharmaceutically acceptable salts” refers to acid addition salts prepared from pharmaceutically acceptable non-toxic acids including inorganic acids and organic acids.

Since fluoxetines and nefazodones are generally basic, salts may be prepared using pharmaceutically acceptable non-toxic acids, including inorganic and organic acids. Such acids include acetic, benzene-sulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic and the like. Particularly preferred are hydrobromic, hydrochloric, phosphoric and sulfuric acids.

The compositions include compositions suitable for oral, rectal, parenteral (including subcutaneous, intramuscular, and intravenous), although the most suitable route in any given case will depend on the nature and severity of the condition being treated. The most preferred route of the present invention is oral. They may be conveniently presented in unit dosage form and prepared by any of the methods well known in the art of pharmacy.

In the case where an oral composition is employed, a suitable dosage range of fluoxetine is, e.g., from about 1 mg to about 50 mg of fluoxetine per day, preferably from about 5 mg to about 45 mg per day and most preferably from about 10 mg to about 40 mg per day, and a suitable dosage range of nefazodone is, e.g., from about 1 mg to about 120 mg of fluoxetine per day, preferably from about 10 mg to about 100 mg per day and most preferably from about 20 mg to about 80 mg per day.

Pharmaceutical carriers suitable for use in subject preparations may be selected according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. The most preferred solid oral preparation is capsules. Because of their ease of administration, tablets and capsules represent the most advantageous oral dosage unit form, in which case solid pharmaceutical carriers are obviously employed. If desired, tablets may be coated by standard aqueous or nonaqueous techniques.

In addition to the common dosage forms set out above, the compounds of the present invention may also be administered by controlled release means and/or delivery devices such as those described in U.S. Pat. Nos. 3,845,770; 3,916,899; 3,536,809; 3,598,123; 3,630,200 and 4,008,719, the disclosures of which are hereby incorporated herein by reference. The use of a racemic mixture of fluoxetine in a sustained release formulation is disclosed and/or claimed in U.S. Pat. Nos. 4,797,286 and 4,847,092.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binner, lubricant, inert diluent, surface active or dispersing agent. Molded tablets may be made by molding in a suitable machine, a mixture of the powdered compound moistened with an inert liquid diluent. In certain embodiments, a dose of about 1 mg to about 50 mg of an SRI and about 1 mg to 120 mg of a nefazodonoid is present in from 1-5 cachets, pills, tablets, or capsules, preferably in one or two such cachets, pills, tablets, or capsules. Most preferably the tablet, cachet, pill, or capsule contains about 10 mg to about 45 mg of fluoxetine and about 10 mg to about 100 mg of nefazodone.

In certain embodiments, the nefazodonoid and the SRI can be chemically linked, e.g., by a linkage that is cleaved (e.g., hydrolyzed) under physiologic conditions. The structure of nefazodone, lacking open valencies on heteroatoms (e.g., OH, NH, SH, etc.), may be modified in order to facilitate such linkage. Although such modifications can affect the biological activities of the component molecules, such modified compounds can be tested individually for biological activity as is well known the art. Such modified variants of nefazodone and fluoxetine are considered “nefazodones” and “fluoxetines”, respectively, for the purposes of these embodiments of the present invention. Furthermore, known metabolites can be employed in the linked compounds as described herein. For example, hydroxynefazodone, which possesses biological activity similar to nefazodone itself, offers an additional hydroxyl which can be used as a site of attachment of a molecular tether without requiring further modification of the molecule.

### IV. Compound Preparation

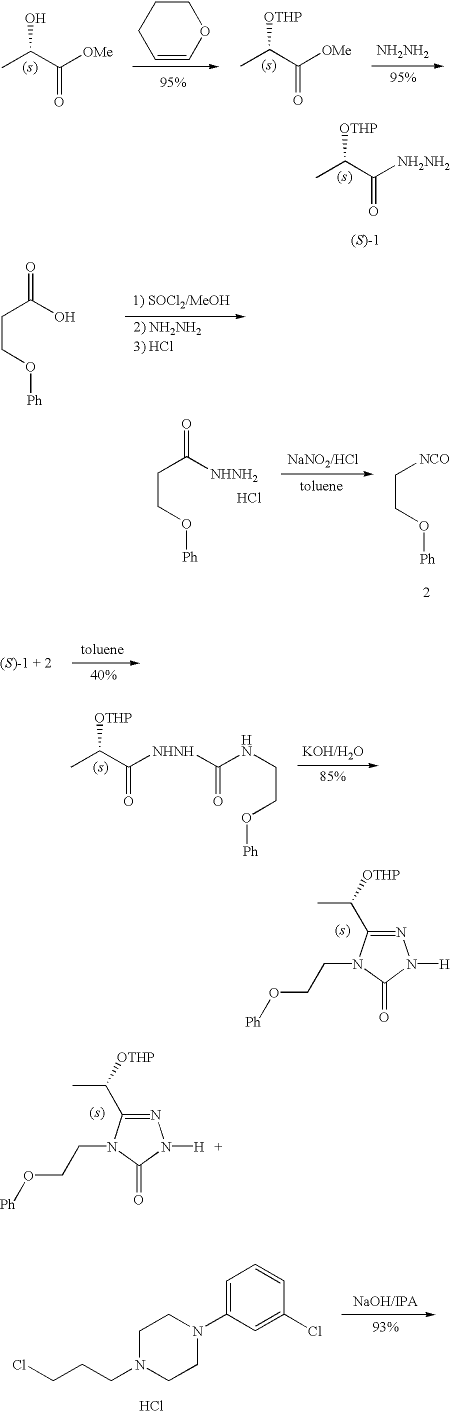

Preparation of the individual enantiomers of hydroxynefazodone is illustrated above and in the following narrative. Alternatively, the R- and S-isomers of hydroxynefazodone may be resolved by methods known to those skilled in the art, for example by formation of diastereoisomeric salts or complexes which may be separated, for example, by crystallisation; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step is required to liberate the desired enantiomeric form.

### V. Exemplary Methods

The present invention contemplates the use of compositions as described above for the treatment or prophylaxis of depression, panic disorder, obsessive compulsive disorders, anxiety, pain (in particular chronic pain), psychoactive substance abuse, migraine headaches, social anxiety/phobic disorder, and posttraumatic stress syndrome, as well as an appetite suppressant. For any of these purposes, treatment includes partial or total alleviation of one or more symptoms of a condition, and prophylaxis includes delaying the onset of or reducing the severity of one or more symptoms of a condition. Although the methods described herein are expected to be effective in any animal, particularly mammals, treatment of humans is preferred in certain embodiments.

The method of the present invention relates to the conjoint administration of a nefazodonoid and an SRI, whether simultaneously, such as in a single composition, or separately, such that a therapeutically effective treatment is achieved for the combination that would not be achieved for either compound alone at the same dosage. Specifically, it is contemplated that a nefazodonoid is administered in an amount sufficient to effectively inhibit 5-HT₂ receptor activity, but not serotonin reuptake, and that an SRI is administered in an amount sufficient to effectively inhibit serotonin reuptake, but not 5-HT₂ receptor activity. In the case where an oral composition is employed, a suitable dosage range of fluoxetine for an adult human is, e.g., from about 1 mg to about 50 mg of fluoxetine per day, preferably from about 5 mg to about 45 mg per day and most preferably from about 10 mg to about 40 mg per day, and a suitable dosage range of nefazodone is, e.g., from about 1 mg to about 120 mg of fluoxetine per day, preferably from about 5 mg to about 100 mg per day and most preferably from about 10 mg to about 80 mg per day. Preferably, nefazodone is administered in an effective amount below about 100 mg/day and fluoxetine is administered in an effective amount below about 50 mg/day. As noted above, one of skill in the art will understand that appropriate dosages will depend, in part, on the age of the patient, the severity of the condition being treated, and other such factors.

The present invention also provides methods of preparing pharmaceutical preparations by combining a nefazodonoid, an SRI, and a pharmaceutically acceptable excipient. Guidance for selecting appropriate compounds and dosages is provided above. The present invention similarly provides for the use of an SRI and a nefazodonoid in a pharmaceutical preparation for the treatment or prophylaxis of depression, panic disorder, obsessive compulsive disorders, anxiety, pain (in particular chronic pain), psychoactive substance abuse, migraine headaches, social anxiety/phobic disorder, and posttraumatic stress syndrome, as well for appetite suppression. In certain such compositions, the SRI, the nefazodonoid, and excipient are intimately mixed or commingled, while in other embodiments, the SRI and nefazodone are substantially separate (e.g., present in distinct layers or portions of a capsule, pill, or tablet).

All of the references and publications cited herein, U.S. Pat. Nos. 6,191,133, 6,143,325, 6,140,323, 6,034,085, 6,001,848, 5,922,341, 5,900,485, 5,885,976, 5,854,248, 5,852,020, 5,788,986, 5,708,035, 5,691,325, 5,691,324, 5,504,086, 4,626,549, 4,596,884, and 4,314,081; PCT Application No. WO 00/61128; and Laird L K. Mood Disorders I: Major Depressive Disorders, in Applied Therapeutics: The Clinical Use of Drugs, 6th edition, Young L Y, Koda-Kimble M A, ed. Applied Therapeutics, Inc., 76-1 -76-25, 1995; Hirshfield R M A. Arch Gen Psychiatry, 29:35, 1982; American Psychiatric Association, Diagnostic and Statistical Manual of Mental Disorders (DSM IV), 4th edition, American Psychiatric Association, DC, 1994; Fontaine R. J Clin Psychiatry, 55:234-241,1994; Eison A S. Psychopharmacol Bull, 26(3):311-315, 1990; Feigner J P. Psychopharmacol Bull, 25(2):219-221, 1989; Taylor D P. BMY 13754-1: Summary of Pharmacologic Activities, Bristol-Meyers Report No: TAYL-DP-11280, 1995; Hamik A. Biol Psychiatry, 25:569-575, 1989; Sharpley A L. Biol Psychiatry; 31:1070-1073, 1992; Vogel G W. Neurosci Biobehav Rev, 14:49-63, 1990; D'Amico M F. Psychopharmacol Bull, 26(1):147-149, 1990; Scott J A. Neuropharmacology, 25:1301-1306, 1986; Fontaine R. Clin Neuropharmacol, 16(Supp.3):S45-S50, 1993; Anonymous, Med Lett Drugs Ther, 37:33, 1995; Drug Facts and Comparisons, Monthly update loose-leaf drug information service, Facts and Comparisons, Inc., St. Louis, Mo., 1996; Anonymous, Nefazodone Monograph In: Poisindex, Micromedex, Inc., Denver, Colo., 1997, Brosen K et al. Br J Clin Pharmacol 1991;32:136-7; Schmidt M J, et al. Br J Psychiatr 1988, 153(suppl 3):40-6; Bergstrom R F, et al. Br J Psychiatr 1988, 153(suppl 3):47-50; Wong D T, et al. Life Sciences 1974, 15:471-9; Altamura A C, et al. Clin Pharmacokinet 1994, 26(3):201-14; Lemberger L, et al. Clin Pharmacol Ther 1978, 23(4):421-9; Fuller R W et al. DT. J Clin Psychopharmacol 1987, 7(6):36S-43S; Schenker S, et al. Clin Pharmacol Ther 1988, 44(3):353-9; Compendium of Pharmaceuticals and Specialties, Prozac® product monograph 1994:1090-3; Aronoff G R, et al. Clin Pharmacol Ther 1984, 36(1):138-44; Lemberger L, et al. J Clin Psychiatry 1985, 46(3):14-9; Fuller R W, et al. Biochem Pharmacol 1978, 27:193-8; Bergstrom R, et al. Abstracts of the American Pharmaceutical Association Academy of Pharmaceutical Sciences 14:110, 1984; Bergstrom R F, et al. Abstracts of the American Pharmaceutical Association Academy of Pharmaceutical Sciences 16:126, 1986a; Gardier A M, et al. Life Sciences 1993, 54:PL51-6; Vaughan D A. Am J Psychiatr 1989, 146(4):562-4; Suckow R F, et al. Clin Chem 1992, 38/9:1756-61; J Clin Psychiatry 2000 Feb;61(2):146; J Psychopharmacol 1997;11(2):190-1; J Psychosoc Nurs Ment Health Serv. 2000 Aug;38(8):20-5; and J Clin Psychiatry 1996;57 Suppl 2:6-9 are hereby incorporated by reference.

### VI. Examplification

### A. Synthesis of (S)— and (R)-Hydroxynefazodone hydrochloride

### EXAMPLE 1

Synthesis of (S)-O-(tetryhdrioyranyl)-methyl lactate

A 250 mL RBF was wquipped with 10.0 g (96 mmol) of (S)-methyl lactate. To the reaction at 23° C. was added 100 mL of CH₂Cl₂, followed by 13.12 (144 mmol) of dihydropyran and a crystal of TsOH. After stirring for 1 h at rt, the reaction mixture was washed with H₂O (2×100 mL). The combuned organic phases were dried (MgSO₄) and concentrated in vacuo to provide 18.2 g of crude product (100%). ¹H NMR (CDCl₃) δ1.41-1.89 (m, 9H), 3.46 (m, 1H), 3.76 (s, 3H), 3.89 (m,1H), 4.34 (m, 1H), 4.72 (m,1H).

### EXAMPLE 2

Synthesis of (S)-O- (tetryhdropyranyl)-methyl lactate hydrozide.

A 100 mL RBF was charged with 18.0 g of (S)-O-(tetryhdropyranyl)-methyl lactate (95.58 mmol). To the reaction mixture was added McOH (25 mL), followed by hydrazine (3.0 mL, 95.58 mmol) at )° C. and the reaction was allowed to stir overnight. The solution was concentrated in vacuo to remove excess hydrazine and the crude adduct was chromatographed with 100% EtOAc to provide 15.2 g of pure product (84%). ¹H NMR (CDCL₃) Γ 1.40-1.91 (m,9H), 3.87 (m,2H), 4.29 (m,1H), 4.64 (m,1H).

### EXAMPLE 3

Synthesis of Methyl 3-phenoxypropionate

3-Phenoxypropionic acid (10.0g, 60 mmol) was dissolved inmethanol (100 mL). The reaction mixture was allowed to cool to 0° C. and SOCL₂ was slowly added over a 15 min period. The reaction mixture was concentrated in bacuo, then redissolved in ehtyl acetate (100 mL). The organic layer was washed with water (2×150 mL), dried (MgSO₄), concentrated in vacuo to provide crude product in 95% yield. ¹H NMR (CDCl₃) Γ 2.84 (t=6.4Hz, 2H), 3.76(s,3H), 3.76(s,3H), 4.28(t,J=6.4Hz,2H), 6.95(m,3H), 7.31 (m,2H).

### EXAMPLE 4

Synthesis of 3-Phenoxypropionyl Hydrazide

A 100 ml round bottom flask was charged with 17.4 g of methyl 3-phenoxypropionate (97.1 mmol). To the reaction mixture was added hydrazine (3.65 mL, 116.5 mmol) at rt and the reaction was allowed to stir overnight. The slurry was concentrated in vacuo to remove excess hydrazine and the product was collected by filtration and washed with hexane (25 mL) to provide 14.9 g (86%) of pure product as an off-white solid. ¹H NMR (CDCl₃) δ 2.67 (t, J=6.0 Hz, 2H), 3.95 (bs, 2H), 4.27 (t, J=6.0 Hz, 2H), 6.94 (m, 3H), 7.29 (m, 2H).

### EXAMPLE 5

### Synthesis of 3-Phenoxypropionyl Hydrazide Hydrochloride

Crude 3-phenoxypropionyl hydrazide (14.6 g, 81.1 mmol) was dissolved in 37 mL of methylene chloride. The solution was stored at 0° C. as anhydrous 1N HCl in ether (89.2 mL, 89.2 mmol) was slowly added. After stirring for 1 h at 0° C., the solid was collected by filtration, rinsed with methylene chloride (2×15.0 mL methylene chloride), and dried in vacuo. The solid weighed 15.2 g (85%). ¹H NMR (DMSO-D₆) δ 2.72 (t, J=5.4 Hz, 2H), 4.20 (t, J=5.4 Hz, 2H), 6.91 (m, 3H), 7.26 (m, 2H).

### EXAMPLE 6

### Synthesis of 2-Phenoxyethyl isocyanate

A slurry of 3-phenoxypropionyl hydrazide hydrochloride (10.0 g, 46.12 mmol), 3.83 mL (46.2 mmol) of 37% HCl, 41.1 mL of H₂O, and 24.1 mL of toluene was stirred in an ice bath as a solution of 3.50 g (50.7 mmol) of sodium nitrate in 14.1 mL of H₂O was added over 20 minutes. The reaction temperature was not allowed to exceed 18° C. After 20 min, the mixture was filtered and the organic phase separated. The aqueous layer was extracted with 8 mL of toluene. The combined organic layers were dried over anhydrous MgSO₄. The dried toluene layer was slowly added over a 1 h period with stirring to an empty flask heated at 85° C. When the addition was complete and nitrogen evolution has stopped (bubbling stops), the solution was cooled to rt. ¹H NMR (CDCl₃) δ 3.65 (t, J=10.2 Hz, 2H), 4.07 (t, J=10.2 Hz, 2H), 6.96 (m, 3H), 7.29 (m, 2H).

### EXAMPLE 7

### Synthesis of 1-((2S)—O-tertahydropyranyl)-propionyl-4-(2-phenoxyethyl)semicarbazide

A 250 mL RBF was charged with 9.55 g of 2-Phenoxyethyl isocyanate (58.6 mmol) and toluene (40 mL). The reaction flask was cooled to 0° C. and charged with neat (S)—O-(tetryhdropyranyl)-methyl lactate hydrazide (11.01 g, 58.6 mmol). The reaction was allowed to slowly warm to rt overnight. The next morning, the solution was concentrated in vacuo and chromatographed with 100% EtOAc to provide 7.3 g (36%) of pure product as an oil. ¹H NMR (CDCl₃) δ 1.40-1.98 (m, 9H), 3.53 (m, 3H), 3.99 (m, 3H), 4.30 (m 1H), 4.64 (m, 1H), 6.17 (m, 1H), 6.89 (m, 3H), 7.25 (m, 2H), 7.86 (bs, 1H), 8.44 (bs, 1H), 8.56 (bs, 1H).

### EXAMPLE 8

### Synthesis of 5-[(1S)-1-(tetrahydropyran-2-yl)oxyethl)-4-(2-phenoxyethyl)-2H-1,2,4-triazol-3(4H)-one

A 250 mL RBF was charged with 7.2 g of 1-((2S)—O—tertahydropyranyl)-propionyl-4-(2-phenoxyethyl) semicarbazide (20.5 mmol). To the reaction mixture was added 110.8 mL of water, followed by solid KOH (1.20 g, 21.5 mmol). The reaction was warmed to 95° C. and allowed to stir for 6 h. The solution was cooled to 0° C. and treated with 37% aqueous HCl solution and 100 mL of dichloromethane. The phases were separated and the organic phase was washed with water, dried (MgSO₄), filtered, and concentrated in vacuo to provide crude product. The product was purified by chromatography with 75% EtOAc/hexane to 100% EtOAc to provide 5.5 g (80%) of pure product. ¹H NMR (CDCl₃) δ 1.52-1.84 (m, 9H), 3.49 (m, 1H), 3.85 (m, 1H), 4.26 (m, 4H), 4.75 (m, 1H), 5.05 (m, 1H), 6.94 (m, 1H), 7.28 (m, 2H), 10.05 (bs, 1H).

### EXAMPLE 9

### Synthesis of (S)-2-{3-[4-(3-Chlorophenyl)-1-piperazinyl]propyl}-4-(2 -phenoxyethyl)-5-[1-(tetrahydropyran-2-yloxy)-ethyl]-2,4-dihydro-[1,2,4]triazol-3-one

A mixture of 40.0 g (120.0 mmol) of 5-((1S)—O—tetrahydropyranyl)-4-(2-phenoxyethyl)-2H-1,2,4-triazol-3(4H)-one, 40.8 g (132.0 mmol) 1-(3-chlorophenyl)-4-(3-chloropropyl)piperazine hydrochloride, 13.91 mL (264.0 mmol) of 50% aqueous sodium hydroxide and 162 mL of 2-propanol was stirred and heated at reflux for 5.5 h. The mixture was filtered hot. The filtrate was concentrated in vacuo and chromatographed with 100% EtOAc to provide 61.1 g (93%) of pure product as an oil. 1H NMR (CDCl₃) δ 1.51 (m, 3H), 1.62 (d, J=9.1 Hz, 3H), 1.71-1.81 (m, 2H), 1.98 (t, J=7.2 Hz, 3H), 2.45 (t, J=7.2 Hz, 3H), 2.56 (m, 4H), 3.18 (m, 4H), 3.49 (m, 1H), 3.87 (m, 3H), 4.25 (m, 4H), 4.74 (m, 1H), 5.02 (m, 1H), 6.87 (m, 6H), 7.16 (m, 1H), 7.25 (m, 2H).

### EXAMPLE 10

### Synthesis of (S)-2-{3-[4-(3-Chlorophenyl)-1-piperazinyl[propyl}-4-(2-phenoxyethyl)-5-[1-(hydroxy)-ethyl]-2,4-dihydro-[1,2,4]triazol-3-one ((S)-hydroxynefazodone)

A solution of 61.0 g (112 mmol) of 2-[3-[4-(3-Chlorophenyl)-1-piperazinyl]propyl]-5-((1S)—O-tetrahydropyranyl)-4-(2-phenoxyethyl)-2H-1,2,4-triazol-3(4H)-one in 350 mL of THF was treated with 350 mL of 3N HCl at rt. After stirring for 1 h, the solution was concentrated in vacuo and treated with 50% aqueous NaOH solution until pH to 10. The aqueous solution was extracted with dichloromethane (400 mL ×2). The organic phase was washed with water (300 mL), dried (MgSO₄) and concentrated in vacuo. The crude adduct was chromatographed with 2% MeOH/EtOAc to 4% MeOH/EtOAc to provide 51.1 g (93 %) of pure product. ¹H NMR (CDCl₃) δ 1.30 (bs, 1H), 1.65 (d, J=10.5 Hz, 3H), 1.99 (t, J=10.2 Hz, 2H), 2.47 (t, J=7.2 Hz, 2H), 2.58 (m, 4H), 3.19 (m, 4H), 3.89 (t, J=10.2 Hz, 2H), 4.18 (m, 1H), 4.26 (m, 3H), 5.05 (q, J=10.5 Hz, 1H), 6.87 (m, 5H), 7.03 (m, 1H), 7.16 (m, 1H), 7.26 (m, 2H).

### EXAMPLE 11

### Synthesis of (S)-2-{3-[4-(3-Chlorophenyl)-1-piperazinyl]propyl}-4-(2-phenoxyethyl)-5-[1-(hydroxy)-ethyl]-2,4-dihydro-[1,2,4]triazol-3-one Hydrochloride ((S)-hydroxynefazodone hydroxychloride)

A solution of 2-[3-[4-(3-chlorophenyl)-1-piperazinyl]propyl]-5-((1S)-hydroxyl)-4-(2-phenoxyethyl)-2H-1,2,4-triazol-3(4H)-one (51.1 g, 104.9 mmol) in 567 mL of MTBE was slowly charged with 78.0 mL (157.0 mmol) of 2N HCl over a 15 min. period. After stirring for 1.5 h at 0° C., the slurry was filtered in vacuo to provide 44.0 g (80%) of (S)-hydroxynefazodone HCl as a white solid. The white solid was dissolved in 49 mL of refluxing IPA and slowly allowed to cool to rt. The white solids were collected by filtration to provide 37.3 g (84% recovery) of (S)-hydroxynefazodone HCl as a white solid (99.39% chemical purity, 98.66% ee). The ee was determined by chiral HPLC (Chiralcel OD, 10 um, 4.6×250 nm, hexane/IPA/MeOH/diethyl amine 85:10:5:0.1, 1 mL/min, 230 nm, ambient temperature, (S)-isomer 12.89 min, (R)-isomer 14.47 min). Optical rotation [α]=−32.81° (c.1.02, MeOH). ¹H NMR (DMSO-D₆) δ 1.47 (bs, 3H), 2.11 (m, 2H), 3.14 (m, 4H), 3.50 (m, 4H), 3.76 (m, 4H), 4.17 (m, 4H), 4.80 (m, 1H), 6.92 (m, 6H), 7.29 (m, 3H). ¹³C: δ 20.62, 23.70; 42.67; 45.50, 51.11, 53:62; 61.24, 65.42, 115.02, 115.92, 119.18, 119.88, 121.63, 124.29, 130.28, 131.32, 133.49, 134.65, 136.07, 149.31, 151.51, 151.57, 154.08, 158.65. MS m/z 485.94. Anal. Calcd for C₂₅H₃₃Cl₂N₅O₃: C, 57.47; H, 6.37; N, 13.40. Found: C, 57.01; H, 6.39; N, 13.38.

### EXAMPLE 12

### Synthesis of (R)-Hydroxynefazodone hydrochloride

(R)-Hydroxynefazodone hydrochloride (29.5 g, 98.5 % ee, 99.42% chemical purity) was prepared from the (R)-methyl lactate followed the procedure described above for the (S)-isomer. [α]=+32.5 (c. 2, MeOH).

### B. Biological Testing

### General Experimental Procedures

Dopamine and serotonin receptor binding assays were performed in a standard manner with the incubation of membrane preparations in an assay buffer in the presence of a known radioactively labeled specific ligand for the receptor subtypes. Nonspecific binding was determined by assessing binding in the presence of excess ligand. Specific binding was measured as the total labeled ligand bound after the nonspecific binding was subtracted. The effect of the tested agents was measured by determining the competition for the receptor binding across a concentration range. Subsequently, an IC₅₀ was determined for the agents tested. More specific details are provided below for several of the assays performed.

### EXAMPLE 13

### Human D₂ Receptor

Aliquots of transfected A9L cell membrane preparations corresponding to 20-40 μg protein are incubated for 60 min at 22° C. in 250 μL of 50 mM Tris-HCl buffer (pH 7.4) containing 120 mM NaCl, 5 mM KCl, 5 mM MgCl₂, 1 mM EDTA, 0.3 nM [³H]spiperone and increasing concentrations of the competing drugs. Nonspecific binding is determined in the presence of 10 μM (+)butaclamol. After incubation, the samples are filtered rapidly under vacuum through glass fiber filters (GF/B, Packard) and rinsed several times with ice-cold 50 mM Tris-HCl using a cell harvester (Packard). Bound radioactivity is measured with a scintillation counter (Topcount, Packard) using a liquid-scintillation cocktail (Microscint 0, Packard). The reference compound for this assay is (+)butaclamol.

### EXAMPLE 14

### Human D_{4.4} Receptor

Aliquots of transfected CHO cell membrane preparations corresponding to 100 μg protein are incubated for 60 min at 22° C. in 250 μL of 50 mM Tris-HCl buffer (pH 7.4) containing 120 mM NaCl, 5 mM KCl,5, mM MgCl₂, 1 mM EDTA, 0.3 nM [³H]spiperone and increasing concentrations of the competing drugs. Nonspecific binding is determined in the presence of 10 μM (+)butaclamol. After incubation, the samples are filtered rapidly under vacuum through glass fiber filters (GF/B, Packard) and rinsed several times with ice-cold 50 mM Tris-HCl using a cell harvester (Packard). Bound radioactivity is measured with a scintillation counter (Topcount, Packard) using a liquid scintillation cocktail (Microscint 0, Packard). The reference compound for this assay is clozapine.

### EXAMPLE 15

### Human 5-HT_{1A} Receptor

Aliquots of transfected CHO cell membrane preparations corresponding to 7-15 μg protein are incubated for 60 min at 22° C. in 250 [tL of 50 mM Tris-HCl buffer (pH 7.4) containing 10 mM MgSO₄, 0.5 mM EDTA, 0.3 nM [³H]8-OH-DPAT and increasing concentrations of the competing drugs. Nonspecific binding is determined in the presence of 10 μM 8-OH-DPAT. After incubation, the samples are filtered rapidly under vacuum through glass fiber filters (GF/B, Packard) and rinsed several times with ice-cold 50 mM Tris-HCl using a cell harvester (Packard). Bound radioactivity is measured with a scintillation counter (Topcount, Packard) using a liquid scintillation cocktail (Microscint 0, Packard). The reference compound for this assay is 8-OH-DPAT.

### EXAMPLE 16

### Human 5-HT_{2A} Receptor

Aliquots of transfected CHO cell membrane preparations corresponding to 20-50 μg protein are incubated for 15 min at 37° C. in 250 μL of 50 mM Tris-HCl buffer (pH 7.4) containing 2 nM [³H]ketanserin and increasing concentrations of the competing drugs. Nonspecific binding is determined in the presence of 1 μM ketanserin. After incubation, the samples are filtered rapidly under vacuum through glass fiber filters.(GF/B, Packard) and-rinsed several times with ice-cold 50 mM Tris-HCl using a cell harvester (Packard). Bound radioactivity is measured with a scintillation counter (Topcount, Packard) using a liquid scintillation cocktail (Microscint 0, Packard). The reference compound for this assay is ketanserin.

### EXAMPLE 17

### Human 5-HT_{2c} Receptor

Aliquots of transfected CHO cell membrane preparations corresponding to 5-10 μg protein are incubated for 30 min at 37° C. in 250 μl of 50 mM Tris-HCl buffer (pH 7.7) containing 10 μM pargyline, 0.7 nM [³H]mesulergine and increasing concentrations of the competing drugs. Nonspecific binding is determined in the presence of 1 μM mesulergine. After incubation, the samples are filtered rapidly under vacuum through glass fiber filters (GF/B, Packard) and rinsed several times with ice-cold 50 mM Tris-HCl using a cell harvester (Packard). Bound radioactivity is measured with a scintillation counter (Topcount, Packard) using a liquid scintillation cocktail (Microscint 0, Packard). The reference compound for this assay is mesulergine.

### EXAMPLE 18

### Human 5-HT₃ Receptor

Aliquots of transfected HEK-293 cell membrane preparations corresponding to 3-5 μg protein are incubated for 60 min at 22° C. in 250 μt of 50 mM Tris-HCl buffer (pH 7.4) containing 5 mM MgCl₂, 1 mM EDTA, 0.5 nM [³H]BRL 43694 and increasing concentrations of the competing drugs. Nonspecific binding is determined in the presence of 10 μM MDL 72222. After incubation, the samples are filtered rapidly under vacuum through glass fiber filters (GF/B, Packard) and rinsed several times with ice-cold 50 mM Tris-HCl using a cell harvester (Packard). Bound radioactivity is measured with a scintillation counter (Topcount, Packard) using a liquid scintillation cocktail (Microscint 0, Packard). The reference compound for this assay is MDL 72222.

### EXAMPLE 19

### Guinea-pig 5-HT₄ Receptor

Aliquots of guinea-pig striatum membrane preparations corresponding to 600 μg protein are incubated for 30 min at 22° C. in 1 ml of 50 mM Hepes-Tris buffer (pH 7.4) containing 0.1 nM [³H]GR 113808 and increasing concentrations of the competing drugs. Nonspecific binding is determined in the presence of 30 μM 5-HT. After incubation, the samples are filtered rapidly under vacuum through glass fiber filters (Filtermat B, Wallac) and rinsed several times with ice-cold 50 mM Hepes-Tris using a cell harvester (Tomtec). Bound radioactivity is measured with a scintillation counter (Betaplate, Wallac) using a solid scintillant (MeltiLex B/HS, Wallac). The reference compound for this assay is 5-HT.

### Experimental Conditions for Monoamine Uptake Assays

### EXAMPLE 20

### Serotonin Uptake Functional Assay

Characterization of serotonin uptake is performed using synaptosomes isolated in a 0.32 M sucrose buffer from a male Wistar rat cortex. The uptake of radiolabelled serotonin by synaptosomes (100 μg of proteins/point) is allowed by incubating them for 15 minutes at 37° C. in presence of test compounds and [3H]5-hydroxytryptamin (0.1 μCi/point). The experiment is performed in a deep well.

Synaptosomes and [3H]5-hydroxytryptamin are prepared in a Krebs buffer pH 7.4 containing 25 mM NaHCO₃, 11 mM glucose and 50 μM ascorbic acid. This incubation buffer is oxygenated during 5 minutes before incubation. Basal control is incubated for 15 minutes at 4° C. in order to avoid any uptake. Following this incubation the uptake is stopped by filtration through an “unifilter 96-wells GFB” Packard plate washed with Krebs buffer containing 25 mM NaHCO₃ in order to eliminate the free [3H]5-hydroxytrptamin. The radioactivity associated to the synaptosomes retained onto the unifilter corresponding to the uptake is then measured with a microplate scintillation counter Topcount, Packard using a scintillation liquid microscint 0, Packard.

The reference compound is imipramin tested at 10 concentrations ranging from 10⁻¹¹ M to 10⁻⁵ M in order to obtain an IC₅₀ value. [See Perovics and Müller “Pharmacological profile of hypericum extract: effect on serotonin uptake by postsynaptic receptors”, Arzeim. Forsch./Drug Res. 45: 1145-1148 (1995).]

### EXAMPLE 21

### Dopamine Uptake Functional Assay

Characterization of dopamine uptake is performed using synaptosomes isolated at Cerep in a 0.32 M sucrose buffer from a male Wistar rat striatum. The uptake of radiolabelled dopamine by synaptosomes (20 μg of proteins/point) is allowed by incubating them for 15 minutes at 37° C. in presence of test compounds and [3H]-dopamine (0.1 μCi/point). The experiment is performed in a deep well. Synaptosomes and [3H]-dopamine are prepared in a Krebs buffer pH 7.4 containing 25 mM NaHCO₃, 11 mM glucose and 50 μM ascorbic acid. This incubation buffer is oxygenated during 5 minutes before incubation. Basal control is incubated for 15 minutes at 4° C. in order to avoid any uptake. Following this incubation the uptake is stopped by filtration through an “unifilter 96-wells GFB” Packard plate washed with Krebs buffer containing 25 mM NaHCO₃ in order to eliminate the free [3H]-dopamine. The radioactivity associated to the synaptosomes retained onto the unifilter corresponding to the uptake is then measured with a microplate scintillation counter Topcount, Packard using a scintillation liquid microscint 0, Packard. The reference compound is GRB12909 tested at 8 concentrations ranging from 10⁻¹¹ M to 10⁻⁶ M in order to obtain an IC₅₀ value. [See Jankowsky et al. “Characterization of sodium-dependent [3H]GBR-12935 binding in brain: a radioligand for selective labeling of the dopamine transport complex.” Journal of Neurochemistry. 46 (4): 1272-1276 (1986).]

### EXAMPLE 22

### Norepinephrine Uptake Functional Assay

Characterization of norepinephrine uptake is performed using synaptosomes isolated at Cerep in a 0.32 M sucrose buffer from a male Wistar rat hypothalamus. The uptake of radiolabeled norepinephrine by synaptosomes (100 μg of proteins/point) is allowed by incubating them for 20 minutes at 37° C. in presence of test compounds and [3H]-norepinephrine (0.1 μCi/point). The experiment is performed in a deep well.

Synaptosomes and [3H]-norepinephrine are prepared in a Krebs buffer pH 7.4 containing 25 mM NaHCO₃, 11 mM glucose and 50 μM ascorbic acid. This incubation buffer is oxygenated during 5 minutes before incubation. Basal control is incubated for 20 minutes at 4° C. in order to avoid any uptake. Following this incubation the uptake is stopped by filtration through an “unifilter 96-wells GFB” Packard plate washed with Krebs buffer containing 25 mM NaHCO₃ in order to eliminate the free [3H]-norepinephrine. The radioactivity associated to the synaptosomes retained onto the unifilter corresponding to the uptake is then measured with a microplate scintillation counter Topcount, Packard using a scintillation liquid microscint 0, Packard.

The reference compound is imipramine tested at 13 concentrations ranging from 10⁻¹¹ M to 10⁻⁵ M in order to obtain an IC₅₀ value. [See Perovics and Müller, op.cit. (1995).]

The results are shown in the following tables:

TABLE 1
Effect of Nefazodone and Hydroxy-Metabolites on Dopamine Receptor
Binding (IC₅₀ Values, nM)

| | D₁ | D₂ | D₃ | D |
|---|---|---|---|---|
| Nefazodone | 1,980 | 716 | 2,240 | 495 |
| (RS)—OH | 2,270 | 1,420 | 2,560 | 1,310 |
| (R)—OH | 1,690 | 1,690 | 3,490 | 1,910 |
| (S)—OH | 2,310 | 788 | 1,910 | 994 |

TABLE 2
Effect of Nefazodone and Hydroxy-Metabolites on Serotonin Receptor
Binding (IC₅₀ Values, nM)*

| | 5- | 5- | 5- | 5- | 5- | 5- | 5- | 5- | 5- |
|---|---|---|---|---|---|---|---|---|---|
| | HT_{1A} | HT_{1B} | HT_{1D} | HT_{2A} | HT_{2B} | HT_{2C} | HT_{5A} | HT₆ | HT₇ |
| Nefazodone | 625 | 1,870 | 925 | 21 | 56 | 43 | 1,560 | 590 | 71 |
| (RS)—OH | 409 | 4,550 | 1,150 | 20 | 34 | 46 | 2,230 | 856 | 61 |
| (R)—OH | 496 | 3,610 | 1,990 | 22 | 28 | 33 | 2,040 | 1,020 | 71 |
| (S)—OH | 249 | 2,840 | 234 | 18 | 41 | 34 | 1,970 | 489 | 60 |

*Inactive on 5HT₄

TABLE 3
Evaluation of Nefazodone and Hydroxy-Metabolites as inhibitors of
CYP450

| | | R—OH- | S—OH- |
|---|---|---|---|
| | Nef | Nef | Nef |
| CYP1A2 | >200 | >200 | >200 |
| CYP2C8 | 48 | 6 | 23 |
| CYP2C9 | 13 | 21 | 20 |
| CYP2C19 | 20 | 64 | >200 |
| CYP2D6 | 2 | 2 | 2 |
| CYP3A4 | 0.2 | 0.2 | 0.2 |
| BFC | | | |
| CYP3A4 | 2.2 | >200 | 3 |
| BZRes | | | |

TABLE 4
Effect of Nefazodone and Hydroxy-Metabolites on α₁ Receptor
Binding and Monoamine Neuronal Transport (IC₅₀ Values, nM)

| | | 5-HT | NE |
|---|---|---|---|
| | Alpha₁ | Uptake | uptake |
| Nefazodone | 306 | 200 | 1,200 |
| (RS)—OH | 381 | 500 | 1,000 |
| (R)—OH | 367 | 640 | 1,200 |
| (S)—OH | 419 | 790 | 1.500 |

As shown in Table 4, racemic hydroxynefazodone and both of its enantiomers are significantly less active than nefazodone in inhibiting serotonin uptake. As shown in Table 2, all three hydroxynefazodones have high affinity for 5HT2A receptors, at which they are antagonists (data not shown). Since anxiety results from a non-optimal balance between inhibition of serotonin uptake and 5HT receptor blockade, superior treatment of psychiatric disorders can be obtained by combining hydroxynefazodone in any isomeric mixture, with an MRI, particularly an SSRI. As can also be seen from Table 4, (S)-hydroxynefazodone is the least active enantiomer in inhibiting serotonin uptake, and from Table 2 (S)-hydroxynefazodone has the highest affinity for 5HT2A receptors. Therefore, the (S) enantiomer would be the preferred enantiomer to employ. In general, optical purity of greater than 90% would be desirable.

Similarly, since (S)-hydroxynefazodone exhibits excellent affinity for the D2 receptor (Table 1), the combination of (S)-hydroxynefazodone with an antipsychotic agent (a D2 antagonist) allows one to modulate the balance between D2 antagonism and 5HT2A antagonism for optimal antipsychotic therapy with minimal side effects.

### EXAMPLE 23

### (S)-Hydroxynefazodone Tablets

Composition per tablet:

| | | |
|---|---|---|
| (S)-hydroxynefazodone | 25 | mg |
| haloperidol | 5 | mg |
| croscarmellose | 60 | mg |
| colloidal silicon dioxide | 8 | mg |
| magnesium stearate | 1 | mg |
| microcrystalline cellulose | 190 | mg |
| croscarmellose | 15 | mg |
| talc | 10 | mg |
| Total | 539 | mg |

The (S)-hydroxynefazodone, haliperidol and silicon dioxide are dry mixed, the first portion of croscarmellose is added and the mixture is further dry mixed. The magnesium stearate is added, dry mixed and the mixture is run through a roller compactor and mill. The resulting dry granulate is mixed with the remaining three ingredients and compressed into tablets.

### EXAMPLE 24

### (S)-Hvdroxvnefazodone Tablets

The ingredients below are mixed well in the proportions shown in a high shear mixer until uniform granules result. The mixture is tray-dried at 40° C. under vacuum until the desired consistency is reached. The granules are milled to less than 60 mesh using a screen mill and compressed into tablets.

Composition per unit dosage:

| | |
|---|---|
| (S)-hydroxynefazodone | 200 mg |
| clozapine | 50 mg |
| pregelatinized starch | 190 mg |
| microcrystalline cellulose | 25 mg |
| povidone | 15 mg |
| croscarmellose | 10 mg |
| magnesium stearate | 3.75 mg |
| FD & C yellow #2 lake | 2.5 mg |
| Water | (5 mL) |
| Total | 496.25 mg |

### EXAMPLE 24

### (S/R)-Hydroxynefazodone Powder-Filled Capsules.

The hydroxynefazodone, fluoxetine, lactose and cornstarch, in the proportions shown below, are blended until uniform and then the magnesium stearate is blended into the resulting powder, which is sieved and filled into suitably sized two-piece, hard gelatin capsules using conventional machinery. Other doses may be prepared by altering the fill weight and, if necessary, changing the capsule size to suit.

Composition per unit dosage:

| | | |
|---|---|---|
| rac-hydroxynefazodone | 200 | mg |
| fluoxetine | 10 | mg |
| lactose | 250 | mg |
| corn starch | 60 | mg |
| magnesium stearate | 5 | mg |

### Equivalents

Those skilled in the art will recognize, or be able to ascertain using no more than routine experimentation, numerous equivalents to the compounds and methods of use thereof described herein. Such equivalents are considered to be within the scope of this invention and are covered by the following claims.

We claim:

1. A pharmaceutical preparation comprising a nefazodonoid and a serotonin reuptake inhibitor (SRI), in a pharmaceutically acceptable excipient.

2. The preparation of claim 1, wherein the nefazodonoid is selected from nefazodone, hydroxynefazodone, oxonefazodone, a mixture thereof, and pharmaceutically acceptable salts thereof.

3. The preparation of claim 1, wherein the nefazodonoid is R-hydroxynefazodone.

4. The preparation of claim 1, wherein the SRI is a compound represented in Formula (IX), or a pharmaceutically acceptable salts thereof: 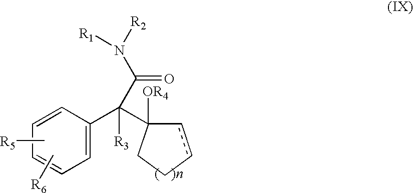
wherein
R₁ is hydrogen or alkyl of 1 to 6 carbon atoms;
R₂ is alkyl of 1 to 6 carbon atoms;
R₃ is hydrogen or alkyl of 1 to 6 carbon atoms;
R₄ is hydrogen, alkyl of 1 to 6 carbon atoms, formyl, or alkanoyl of 2 to 7 carbon atoms;
R₅ and R₆ are independently hydrogen, hydroxyl, alkyl of 1 to 6 carbon atoms, alkoxy of 1 to 6 carbon atoms, alkanoyloxy of 2 to 7 carbon atoms, cyano, nitro, alkylmercapto of 1 to 6 carbon atoms, amino, alkylamino of 1 to 6 carbon atoms, dialkylamino in which each alkyl group is of 1 to 6 carbon atoms, alkanamido of 2 to 7 carbon atoms, halo, trifluoromethyl, or, when taken together, methylene dioxy; and
n is one of the integers 0, 1, 2, 3 or 4.

5. The preparation of claim 1, wherein the SRI is a selective serotonin reuptake inhibitor (SSRI).

6. The preparation of claim 5, wherein the SSRI is a fluoxetinoid.

7. The preparation of claim 5, wherein the SSRI is a compound having a structure represented in formula (III), or a pharmaceutically acceptable salts thereof: 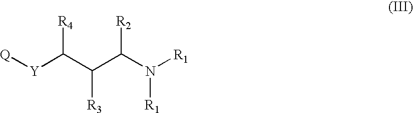
wherein, as valence and stability permit,
R₁, independently for each occurrence, represents H or lower alkyl, preferably H or Me;
R₂, R₃, and R₄ each independently represent H, methyl, substituted or unsubstituted phenyl, or substituted or unsubstituted phenylmethyl, such that exactly one of R₂, R₃, and R₄ is a substituted or unsubstituted phenyl, or substituted or unsubstituted phenylmethyl;
Y represents O, S, or —S(O)₂—, preferably O;
Q represents a substituted or unsubstituted aryl or heteroaryl ring.

8. The preparation of claim 6, wherein the fluoxetinoid is selected from fluoxetine and norfluoxetine, a mixture thereof, and pharmaceutically acceptable salts thereof.

9. The preparation of claim 8, wherein the SSRI is R-fluoxetine.

10. The preparation of claim 5, wherein the SSRI is a compound having a structure represented in formula (V), or a pharmaceutically acceptable salts thereof: 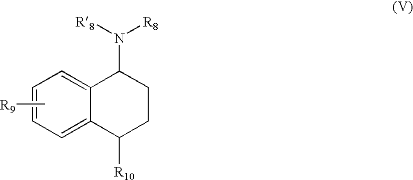
wherein
R₈ is selected from the group consisting of hydrogen and normal alkyl of from 1 to 3 carbon atoms;
R′₈ is normal alkyl of from 1 to 3 carbon atoms;
R₉ is selected from the group consisting of hydrogen, fluoro, chloro, bromo, trifluoromethyl and alkoxy of from 1 to 3 carbon atoms;
R₁₀ is 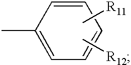
R₁₁ and R₁₂ are each independently selected from the group consisting of hydrogen, fluoro, chloro, bromo, trifluoromethyl, alkoxy of from 1 to 3 carbon atoms and cyano, with at least one of R₁₁ and R₁₂ being other than hydrogen.

11. The preparation of claim 5, wherein the SSRI is a compound having a structure represented in formula (VI), or a pharmaceutically acceptable salts thereof: 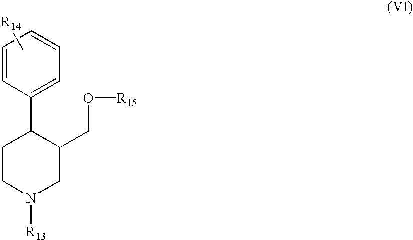
wherein
R₁₃ represents hydrogen or an alkyl group of 1-4 carbon atoms, and
R₁₄ represents hydrogen, alkyl having 1-4 carbon atoms, C1-6 alkoxy, C1-6 trifluoroalkyl (preferably, trifluoromethyl), hydroxy, halogen, methylthio, or C1-6 aryl(C1-6) alkyloxy (e.g., phenyl(C1 -6)alkyloxy and benzyl(C1-6)alkyloxy), and
R₁₅ represents an alkyl or alkynyl group having 1-4 carbon atoms, or a phenyl group optionally substituted by C1-4 alkyl, C1-6 alkylthio, C1-6 alkoxy, halogen, nitro, acylamino, methylsulfonyl or methylenedioxy, or represents tetrahydronaphthyl.

12. The preparation of claim 5, wherein the SSRI is a compound having a structure represented in formula (VII), or a pharmaceutically acceptable salts thereof: 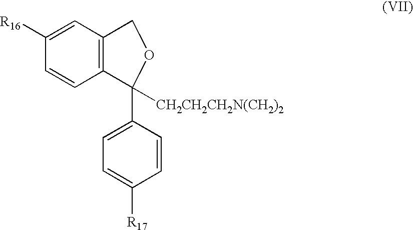
wherein R₁₆ and R₁₇ are each independently represent a halogen, a trifluoromethyl group, a cyano group or —C(═O)—R₁₈, wherein R₁₈ is an alkyl radical with from 1-4 C-atoms inclusive.

13. The preparation of claim 5, wherein the SSRI is a compound having a structure represented in formula (VIII), or a pharmaceutically acceptable salts thereof: 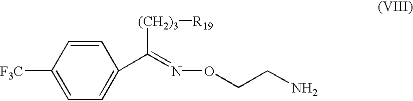
wherein R₁₉ represents a cyano group, a cyanomethyl group, a methoxymethyl group or an ethoxymethyl group.

14. The preparation of claim 1, formulated for oral administration.

15. The preparation of claim 1, wherein the nefazodonoid and SRI are commingled in single dosage form.

16. The preparation of claim 1, wherein the nefazodonoid and SRI are provided in separate dosage form.

17. The preparation of any of claims 1-16, wherein the nefazonoid is provided in an amount, for single dosage, to reach the ED₅₀ for 5-HT receptor inhibition, but less than half the ED₅₀ for inhibition of serotonin reuptake.

18. The preparation of claim 17, wherein the SRI is provided in an amount, for single dosage, to reach the ED₅₀ for inhibition of serotonin reuptake, but less than half the ED₅₀ for 5-HT receptor inhibition.

19. A pharmaceutical preparation comprising, in a single dosage form, a mixture of a nefazodonoid and a fluoxetinoid.

20. The pharmaceutical preparation of claim 19, wherein the nefazodonoid is selected from nefazodone, hydroxynefazodone, oxonefazodone, a mixture thereof, and pharmaceutically acceptable salts thereof.

21. The pharmaceutical preparation of claim 20, wherein the single dosage form contains from 10-100 mg nefazodone, hydroxynefazodone or oxonefazodone.

22. The pharmaceutical preparation of claim 20, wherein the single dosage form contains less than 50 mg nefazodone, hydroxynefazodone or oxonefazodone.

23. The pharmaceutical preparation of claim 19, wherein the single dosage form contains from 5-40 mg fluoxetine or norfluoxetine.

24. The pharmaceutical preparation of claim 19, wherein the single dosage form contains less than 20 mg fluoxetine and norfluoxetine.

25. A kit comprising
a. in single dosage form, a nefazodonoid and a selective serotonin reuptake inhibitor, each in a pharmaceutically acceptable excipient;
b. instructions for co-administering the nefazodonoid and a selective serotonin reuptake inhibitor in a treatment of a serotonin-mediated disorder.

26. A method for treating a 5-HT receptor-mediated disorder in an animal, comprising co-administering to the animal
an amount of a nefazodonoid sufficient to inhibit a 5-HT₂ receptor activity to a therapeutically effective extent, and
an amount of a serotonin reuptake inhibitor (SRI) sufficient to inhibit serotonin reuptake to a therapeutically effective extent,
wherein the nefazodonoid is administered at a dosage below the necessary dosage to inhibit serotonin reuptake to a therapeutically effective extent in the absence of the SRI.

27. The method of claim 26, wherein the nefazodonoid and the SRI are administered simultaneously.

28. The method of claim 27, wherein the nefazodonoid and the SRI are administered as part of a single composition.

29. The method of claim 28, wherein the single composition is for oral administration.

30. The method of claim 26, wherein the nefazodonoid is selected from nefazodone, hydroxynefazodone, oxonefazodone, a mixture thereof, and pharmaceutically acceptable salts thereof.

31. The method of claim 30, wherein the nefazodonoid is R-hydroxynefazodone.

32. The method of claim 26, 30 or 31, wherein the SRI is a fluoxetinoid.

33. The method of claim 32, wherein the fluoxetinoid is selected from fluoxetine and norfluoxetine, a mixture thereof, and pharmaceutically acceptable salts thereof.

34. The method of claim 32, wherein the SSRI is R-fluoxetine.

35. A method for treating depression in a human patient, comprising administering to the patient (a) a nefazodonoid selected from nefazodone, hydroxynefazodone, or oxonefazodone in an amount of 100 mg or less per day, and (b) a fluoxetinoid selected from fluoxetine or norfluoxetine in an amount sufficient to inhibit serotonin reuptake to a therapeutically effective extent.

36. The method of claim 35, wherein the nefazodonoid and the fluoxetinoid are administered to the patient simultaneously.

37. The method of claim 35, wherein the fluoxetinoid is administered at a rate of 5-40 mg per day.

38. The method of claim 35, wherein the nefazodonoid is administered at a rate of less than 50 mg per day.

39. A method for preparing a pharmaceutical preparation, comprising combining a nefazodonoid, a fluoxetinoid, and a pharmaceutically acceptable excipient in a composition for simultaneous administration of the nefazodonoid and the fluoxetinoid.

40. A pharmaceutical preparation of a nefazodonoid and a fluoxetinoid for use in the treatment of a 5-HT receptor mediated disorder.

41. A method for conducting a pharmaceutical business, comprising:
a. manufacturing a preparation of claim 1 or a kit of claim 25; and
b. marketing to healthcare providers the benefits of using the preparation or kit in the treatment of 5-HT receptor-mediated disorders.

42. A method for conducting a pharmaceutical business, comprising:
a. providing a distribution network for selling the preparation of claim 1 or the kit of claim 25; and
b. providing instruction material to patients or physicians for using the preparation to treat 5-HT receptor-mediated disorders.

43. A method for conducting a pharmaceutical business, comprising:
a. determining an appropriate formulation and dosage of a nefazodonoid and a selective serotonin reuptake inhibitor to be co-administered in the treatment of a 5-HT receptor mediated disorder;
b. conducting therapeutic profiling of formulations identified in step (a), for efficacy and toxicity in animals; and
c. providing a distribution network for selling a preparation identified in step (b) as having an acceptable therapeutic profile.

44. The method of claim 43, including an additional step of providing a sales group for marketing the preparation to healthcare providers.

45. A method for conducting a pharmaceutical business, comprising:
a. determining an appropriate formulation and dosage of a nefazodonoid and a selective serotonin reuptake inhibitor to be co-administered in the treatment of a 5-HT receptor mediated disorder; and
b. licensing, to a third party, the rights for further development and sale of the formulation.

46. A single dosage formulation of having 10-50mg of nefazodone, hydroxynefazodone oroxonefazodone, or a mixture thereof.
